(19) United States
(12) Patent Application Publication
(10) Pub. No.: US 2024/0309465 A1
(43) Pub. Date: Sep. 19, 2024

(54) DETECTION METHOD OF AMMONIA OXIDATION FUNCTIONAL GENE IN WATER TREATMENT

(71) Applicant: Chung Yuan Christian University, Taoyuan City (TW)

(72) Inventors: Yu-Tzu Huang, Taoyuan City (TW); Jui-Chuang Wu, Taoyuan City (TW); Ting-Cih Huang, Taoyuan City (TW)

(73) Assignee: Chung Yuan Christian University, Taoyuan City (TW)

(21) Appl. No.: 18/322,595

(22) Filed: May 24, 2023

(30) Foreign Application Priority Data
Mar. 14, 2023 (TW) 112109378

(51) Int. Cl. C12Q 1/689; C12Q 1/6844; G01N 33/53; G01N 33/543

(52) U.S. Cl. CPC C12Q 1/689; C12Q 1/6844; G01N 33/5308; G01N 33/54388; C12Q 1/6804

(57) ABSTRACT

The invention provides a detection method of ammonia oxidation functional gene in water treatment, including the following steps. For a sample to be detected, ammonia oxidation functional genes are amplified by PCR, and primers used for PCR are modified by attaching biotin and digoxigenin to forward and reverse 5′ ends respectively. Next, on the basis of immunochemistry, antibodies are used for biorecognition, and nano colloidal gold particles are used as chromogenic substances. The sample is placed on a sample pad of a lateral flow immunoassay test strip, and the sample flows from the sample pad to an absorbent pad. Color development on both the test and control lines when the sample flows through a nitrocellulose membrane indicates a positive result; color development solely on the control line indicates a negative result and no color development indicates an invalid test.

### CROSS-REFERENCE TO RELATED APPLICATION

This application claims the priority benefit of Taiwan application serial no. 112109378, filed on Mar. 14, 2023. The entirety of the above-mentioned patent application is hereby incorporated by reference herein and made a part of this specification.

### BACKGROUND OF THE INVENTION

### Field of the Invention

The invention relates to a detection method, and in particular to a rapid detection method of ammonia oxidation functional gene in water treatment.

### Description of Related Art

With the development of science and technology, water pollution is becoming more significant. The wastewater discharged by high-tech industries contains high concentrations of ammonia nitrogen, which seriously pollutes rivers. Therefore, sewage treatment is very important. At present, various efficient sewage treatment techniques have been developed, such as physical, chemical, and biological methods. Although the physical and chemical sewage treatment system may bring good results, the cost of treatment due to the large amount of wastewater is not in line with the benefits.

In recent years, the bioreactor of the anammox process has gradually received attention, and the system may efficiently treat ammonia nitrogen wastewater at low cost. However, due to the inability to immediately analyze the distribution and metabolism of microorganisms in the bioreactor, the operator may not accurately control the reactor, thereby increasing operating costs, and blind correction may cause the bioreactor to be paralyzed and lose the effect thereof. Therefore, popularization in practical application may not be achieved. Molecular biological detection techniques are very important for the practical application of the anammox bioreactor, and there are currently many such techniques. Examples include real-time polymerase chain reaction (qPCR), microarray chip, and next-generation sequencing (NGS), etc. These methods may accurately analyze the microorganisms in the bioreactor, but have relatively higher technical threshold and require sophisticated equipment.

Based on the above, a biological detection method for analyzing the bacterial phase of the bioreactor is developed, which may speed up the analysis of the bacterial phase in the bioreactor in the operation of the wastewater treatment plant, detect immediately in the field and know issues in read-time, thereby reducing operating costs, and is an important subject of current research.

### SUMMARY OF THE INVENTION

The invention provides a method of detecting ammonia oxidation functional gene in water treatment. Via a rapid detection test strip of a lateral flow immunoassay method, the result that may be interpreted by naked eyes may be produced within 15 minutes, which may provide the operator with real-time information on the bacteria species for the biological treatment process of high-concentration nitrogen-containing wastewater.

A method of detecting ammonia oxidation functional gene in water treatment of the invention includes the following steps. For a sample to be detected, ammonia oxidation functional genes are amplified by PCR, and primers used for PCR are modified by attaching biotin and digoxigenin to forward and reverse 5′ ends respectively. Then, a lateral flow immunoassay test strip is provided, including a sample pad, a binding pad, a nitrocellulose membrane, an absorbent pad, and a bottom card. The binding pad is configured with a chromogenic substance, and the chromogenic substance is a complex of nano colloidal gold particles and a primary antibody. According to a flow direction of the sample, a test line and a control line are sequentially drawn on the nitrocellulose membrane. A reagent used in the test line is streptavidin, and the control line is configured with a secondary antibody. The absorbent pad is used to provide a capillary attraction and collect the remaining sample. The sample pad, the binding pad, the nitrocellulose membrane, and the absorbent pad are sequentially disposed on the bottom card according to the flow direction of the sample. Next, the sample is placed on the sample pad of the lateral flow immunoassay test strip, and the sample flows from the sample pad to the absorbent pad. Color development on both the test and control lines when the sample flows through the nitrocellulose membrane indicates a positive result; color development solely on the control line indicates a negative result and no color development indicates an invalid test.

In an embodiment of the invention, the primary antibody includes a mouse anti-digoxigenin antibody.

In an embodiment of the invention, the secondary antibody includes a goat anti-mouse antibody.

In an embodiment of the invention, the ammonia oxidation functional gene includes a 16S rRNA of ammonia oxidizing microorganism, nitrite oxidizing bacteria, denitrifying bacteria, or anammox bacteria.

In an embodiment of the invention, a detection concentration of the sample is 1 ng to 0.1 fg.

In an embodiment of the invention, the sample is placed on the sample pad in an amount of 1 ng to 0.1 fg.

In an embodiment of the invention, a material of the sample pad includes a non-woven fabric, a material of the binding pad includes a non-woven fabric, a material of the absorbent pad includes cotton, and a material of the bottom card includes a plastic and an adhesive material.

In an embodiment of the invention, in the flow direction of the sample, a length of the sample pad is 9 mm to 19 mm, a length of the binding pad is 6 mm to 12 mm, a length of the nitrocellulose membrane is 17 mm to 33 mm, a length of the absorbent pad is 14 mm to 26 mm, and a length of the bottom card is 41 mm to 77 mm.

In an embodiment of the invention, the sample pad, the binding padm the nitrocellulose membrane, the absorbent pad, and the bottom card have the same width in the flow direction perpendicular to the sample, with a width of 2 mm to 6 mm.

Based on the above, the invention provides a method of detecting ammonia oxidation functional gene in water treatment, which is developed by lateral flow immunoassay and establishes a rapid biological detection method for analyzing the bacterial phase of a bioreactor, wherein specific primers for the target gene are designed, and the primers are modified for PCR amplification. After the amplification is successful, the lateral flow immunoassay test strip is used for detection, which may be read by naked eyes and semi-quantitative analyzed within 15 minutes, with a sensing limit in the range of 1 ng to 0.1 fg. In this way, the method of detecting ammonium oxidation functional gene in water treatment of the invention may be assisted by the rapid detection test strip, and the analysis of the bacterial phase in the bioreactor may be accelerated in the operation of the wastewater treatment plant to immediately detect and know issues in the field, thus reducing operating costs. In addition, the activity of bacteria may be preliminarily determined for the anammox reactor, so as to avoid blind modification and making the bioreactor become paralyzed and lose the function thereof.

### BRIEF DESCRIPTION OF THE DRA WINGS

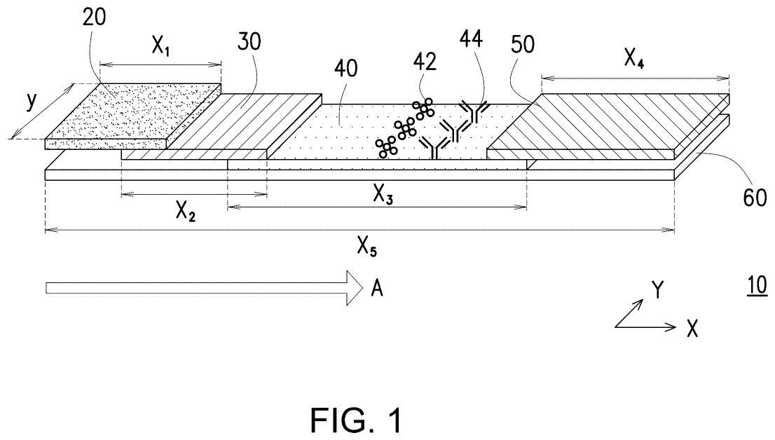

FIG. 1 is a schematic structural diagram of a lateral flow immunoassay test strip according to an embodiment of the invention.

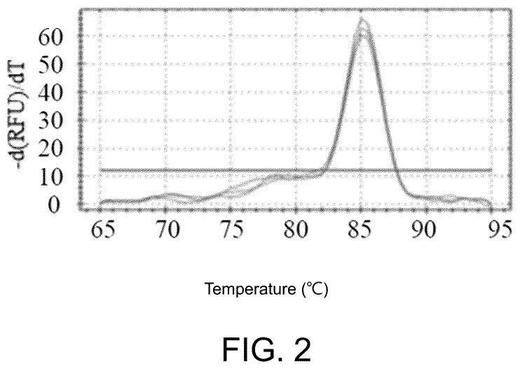

FIG. 2 is a dissociation curve analysis diagram of PCR fluorescent signal for an Anammox-II primer of the invention.

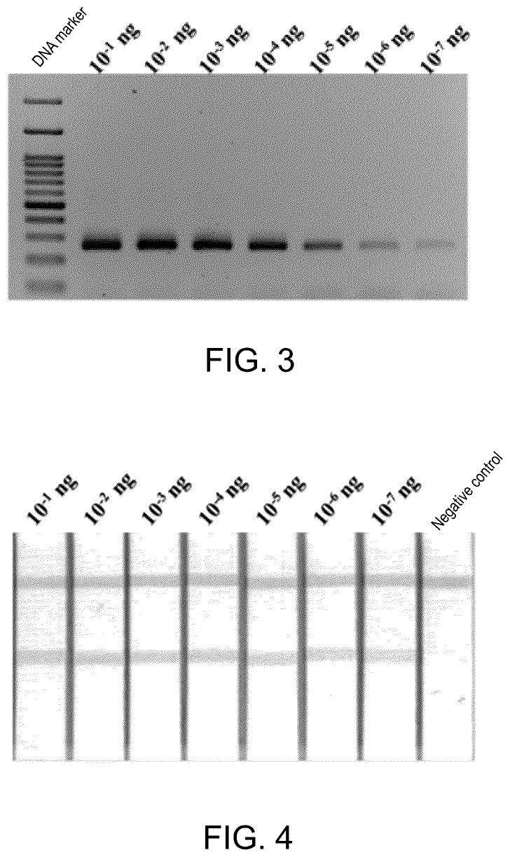

FIG. 3 is an analysis diagram of a detection limit value test of agarose gel electrophoresis of the invention.

FIG. 4 is an analysis diagram of the detection signal of a PCR product amplified by an Anammox-II primer of the invention on a lateral flow immunoassay test strip.

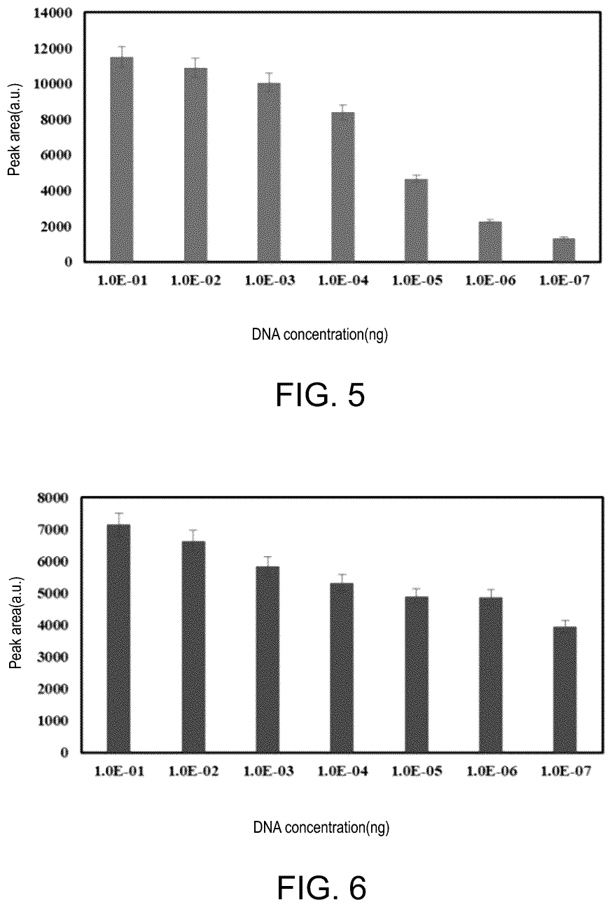

FIG. 5 is an analysis diagram of the signal intensity of an agarose gel electrophoresis result of the invention.

FIG. 6 is an analysis diagram of the signal intensity of a lateral flow immunoassay test strip result.

### DESCRIPTION OF THE EMBODIMENTS

Hereinafter, embodiments of the invention are described in detail. However, these embodiments are illustrative, and the disclosure of the invention is not limited thereto.

Herein, a range indicated by “one value to another value” is a general representation which avoids enumerating all values in the range in the specification. Therefore, the record of a specific numerical range covers any number within this numerical range and any smaller numerical range bounded by any number within that numerical range as if such any number and such smaller numerical ranges were expressly written in the specification.

The technical content, features, and effects related to the invention are clearly presented in the following detailed descriptions of various embodiments with reference to the drawings. The directional terms mentioned in the following embodiments, such as: “up”. “down”. “front”, “back”. “left”. “right”, “bottom”, “top”, etc., are only reference directions of the figures. Accordingly, the directional terms used are illustrative, not limiting, of the invention. In this specification and each drawing, the same reference numeral represents the same element. In the drawings, the size of structures is exaggerated for clarity of the invention.

The detection method of ammonia oxidation functional gene in water treatment of the invention mainly includes the following steps. First, for a sample to be detected, ammonia oxidation functional genes are amplified by polymerase chain reaction (PCR). Next, a lateral flow immunoassay test strip is provided, the sample is placed on a sample pad of the lateral flow immunoassay test strip, and the sample flows from the sample pad to an absorbent pad. Color development on both the test and control lines when the sample flows through the nitrocellulose membrane indicates a positive result; color development solely on the control line indicates a negative result and no color development indicates an invalid test. Hereinafter, the details of each step in the detection method of ammonia oxidation functional gene in water treatment of the invention are described in detail.

### Polymerase Chain Reaction (PCR)

In the detection method of ammonia oxidation functional gene in water treatment of the invention, first, for a sample to be detected, ammonia oxidation functional genes are amplified by PCR, and primers used for the PCR are modified by attaching biotin and digoxigenin to forward and reverse 5′ ends respectively. In the present embodiment, the ammonia oxidation functional genes may include a 16S rRNA of ammonia oxidizing microorganism, nitrite oxidizing bacteria, denitrifying bacteria, or anammox bacteria, and the 16S rRNA is amplified by PCR using the functional gene primer Anammox-II (anaerobic ammonium oxidation, anammox bacteria).

In the present embodiment, for example, PCR conditions are: total reaction volume 10 μL (2× SuperRed PCR Master Mix: 5 μL, forward primer and reverse primer: 0.4 μL each, DNA to be detected: 1 μL, ddH2O: 3.2 μL); reaction conditions: initial denaturation at 95° C. for 1 minute; denaturation at 95° C. for 30 seconds, annealing at 62° C. for 30 seconds, extension at 72° C. for 1 minute, 30 cycles; denaturation at 72° C. for 5 minutes. However, the invention is not limited thereto, and PCR conditions may also be adjusted according to actual operation requirements.

### Lateral Flow Immunoassay Test Strip

FIG. 1 is a schematic structural diagram of a lateral flow immunoassay test strip according to an embodiment of the invention.

Please refer to FIG. 1, a lateral flow immunoassay test strip 10 of the invention includes a sample pad 20, a binding pad 30, a nitrocellulose membrane 40, an absorbent pad 50, and a bottom card 60. The binding pad 30 is configured with a chromogenic substance, the chromogenic substance is a complex of nano colloidal gold particles and a primary antibody, and the primary antibody may include a mouse anti-digoxigenin antibody. The nitrocellulose membrane 40 draws a test line 42 and a control line 44 sequentially on the nitrocellulose membrane 40 according to a flow direction A of the sample. The reagent used in the test line 42 is streptavidin. The control line 44 is configured with a secondary antibody, and the secondary antibody may include a goat anti-mouse antibody. The absorbent pad 50 is used to provide a capillary attraction and collect the remaining sample. The sample pad 20, the binding pad 30, the nitrocellulose membrane 40, and the absorbent pad 50 are sequentially disposed on the bottom card 60 according to the flow direction A of the sample. After the PCR product is obtained via PCR, the lateral flow immunoassay test strip 10 is provided, and the sample is placed on the sample pad 20 of the lateral flow immunoassay test strip 10. The amount of the sample placed on the sample pad 20 is, for example. 1 ng to 0.1 fg. The sample flows from the sample pad 20 to the absorbent pad 50. Color development on both the test and control lines when the sample flows through the nitrocellulose membrane 40 indicates a positive result; color development solely on the control line indicates a negative result and no color development indicates an invalid test.

In the present embodiment, the material of the sample pad 20 may include non-woven fabric, the material of the binding pad 30 may include non-woven fabric, the material of the absorbent pad 50 may include cotton, and the material of the bottom card 60 may include plastic and adhesive material. However, the invention is not limited thereto.

In the present embodiment, in the flow direction of the sample, the length of the sample pad is 9 mm to 19 mm, the length of the binding pad is 6 mm to 12 mm, the length of the nitrocellulose membrane is 17 mm to 33 mm, the length of the absorbent pad is 14 mm to 26 mm, and the length of the bottom card is 41 mm to 77 mm. The sample pad, the binding pad, the nitrocellulose membrane, the absorbent pad, and the bottom card have the same width in the flow direction perpendicular to the sample, with a width of 2 mm to 6 mm. However, the invention is not limited thereto.

### Experimental Examples

FIG. 2 is a dissociation curve analysis diagram of PCR fluorescent signal for an Anammox-II primer of the invention. In this study. PCR reaction fluorescent signal dissociation curve analysis was performed on the Anammox-II primer to test the specificity of the primer. The results show that the primer presents a single peak and has good specificity.

FIG. 3 is an analysis diagram of a detection limit value test of agarose gel electrophoresis of the invention. FIG. 4 is an analysis diagram of the detection signal of a PCR product amplified by an Anammox-II primer of the invention on a lateral flow immunoassay test strip. FIG. 5 is an analysis diagram of the signal intensity of an agarose gel electrophoresis result of the invention. FIG. 6 is an analysis diagram of the signal intensity of a lateral flow immunoassay test strip result. In addition to availability and specificity in the application of the primer, the detection limit was tested by agarose gel electrophoresis. The purified gene was serially diluted 10 times as a template, so that the gene template concentration ranged from 0.1 ng to 0.1 fg. It may be known from FIG. 3 that the detection limit value may reach at least 0.1 fg. From the electrophoresis results in FIG. 3, it may be observed that as the concentration of the gene template is decreased, the signal intensity presented on the electrophoresis gel is also decreased. The signal change trend of the lateral flow immunoassay test strip observed in FIG. 4 is more stable than that of the agarose gel electrophoresis signal of FIG. 3.

Based on the above, the invention provides a method of detecting ammonia oxidation functional gene in water treatment, which is developed by lateral flow immunoassay and establishes a rapid biological detection method for analyzing the bacterial phase of a bioreactor, wherein specific primers for the target gene are designed, and the primers are modified for PCR amplification. After the amplification is successful, the lateral flow immunoassay test strip is used for detection, which may be observed and rapidly read by naked eyes and semi-quantitative analyzed within 15 minutes, with a sensing limit in the range of 1 ng to 0.1 fg. After the actual analysis of the bioreactor, the difference in gene expression is correlated with the change of water quality, which may preliminarily interpret the general situation of the bioreactor.

The invention provides a method of detecting ammonia oxidation functional gene in water treatment that may immediately analyze the distribution and metabolism of microorganisms in a bioreactor, so that the operator may accurately control the reactor and may immediately detect and know issues in the field. Thereby, operating costs are reduced, and the risk of paralyzing the bioreactor due to blind correction is avoided. Therefore, the popularity of practical application of the bioreactor of the anammox process may be promoted. Moreover, the method of detecting ammonia oxidation functional gene in water treatment of the invention mainly adopts a lateral flow immunoassay test strip for detection, which may be quickly interpreted and semi-quantitatively analyzed by naked eyes within 15 minutes, which is different from the molecular biological detection technique in the prior art, and the shortcomings of high technical threshold and the need for sophisticated equipment may be effectively alleviated.

What is claimed is:

1. A detection method of ammonia oxidation functional gene in water treatment, comprising:
amplifying ammonia oxidation functional genes by a polymerase chain reaction (PCR) and modifying primers used for the PCR by attaching biotin and digoxigenin to forward and reverse 5′ ends respectively for a sample to be detected;
providing a lateral flow immunoassay test strip, comprising:
a sample pad;
a binding pad configured with a chromogenic substance, and the chromogenic substance is a complex of nano colloidal gold particles and a primary antibody;
a nitrocellulose membrane, wherein according to a flow direction of the sample, a test line and a control line are sequentially drawn on the nitrocellulose membrane, a reagent used in the test line is streptavidin, and the control line is configured with a secondary antibody;
an absorbent pad used to provide a capillary attraction and collect the remaining sample; and
a bottom card, wherein the sample pad, the binding pad, the nitrocellulose membrane, and the absorbent pad are sequentially disposed on the bottom card according to the flow direction of the sample; and
placing the sample on the sample pad of the lateral flow immunoassay test strip, wherein the sample flows from the sample pad to the absorbent pad, and color development on both the test and control lines when the sample flows through the nitrocellulose membrane indicates a positive result; color development solely on the control line indicates a negative result and no color development indicates an invalid test.

2. The method of detecting ammonia oxidation functional gene in water treatment of claim 1, wherein the primary antibody comprises a mouse anti-digoxigenin antibody.

3. The method of detecting ammonia oxidation functional gene in water treatment of claim 1, wherein the secondary antibody comprises a goat anti-mouse antibody.

4. The method of detecting ammonia oxidation functional gene in water treatment of claim 1, wherein the ammonia oxidation functional gene comprises a 16S rRNA of ammonia oxidizing microorganism, nitrite oxidizing bacteria, denitrifying bacteria, or anammox bacteria.

5. The method of detecting ammonia oxidation functional gene in water treatment of claim 1, wherein a detection concentration of the sample is 1 ng to 0.1 fg.

6. The method of detecting ammonia oxidation functional gene in water treatment of claim 1, wherein the sample is placed on the sample pad in an amount ranging from 1 ng to 0.1 fg.

7. The method of detecting ammonia oxidation functional gene in water treatment of claim 1, wherein a material of the sample pad comprises a non-woven fabric, a material of the binding pad comprises a non-woven fabric, a material of the absorption pad comprises a cotton, and a material of the bottom card comprises a plastic and an adhesive material.

8. The method of detecting ammonia oxidation functional gene in water treatment of claim 1, wherein in the flow direction of the sample, a length of the sample pad is 9 mm to 19 mm, a length of the binding pad is 6 mm to 12 mm, a length of the nitrocellulose membrane is 17 mm to 33 mm, a length of the absorbent pad is 14 mm to 26 mm, and a length of the bottom card is 41 mm to 77 mm.

9. The method of detecting ammonia oxidation functional gene in water treatment of claim 1, wherein a width of the sample pad, the binding pad, the nitrocellulose membrane, the absorbent pad, and the bottom card in a flow direction perpendicular to the sample is the same, and the width is 2 mm to 6 mm.
